(19) United States
(12) Patent Application Publication
(10) Pub. No.: US 2011/0081420 A1
(43) Pub. Date: Apr. 7, 2011

(54) METHOD OF FORMING PROLONGED-RELEASE INJECTABLE STEROIDS

(75) Inventor: Thomas Harry Barrows, Austell, GA (US)

(73) Assignee: ZYGA TECHNOLOGY, INC., Minneapolis, MN (US)

(21) Appl. No.: 12/772,358

(22) Filed: May 3, 2010

Related U.S. Application Data
(60) Provisional application No. 61/249,415, filed on Oct. 7, 2009.

(51) Int. Cl. A61K 31/58; A61K 31/573; A61K 9/14

(52) U.S. Cl. 424/499; 514/179; 514/174

(57) ABSTRACT

A method of forming prolonged-release injectable steroids. The method includes providing a steroid composition, a bioabsorbable polymer and a solvent. A solution is formed from the steroid composition, the bioabsorbable polymer and the solvent. Droplets are formed from the solution. The solvent is removed from the droplets to cause the droplets to form microspheres.

### REFERENCE TO RELATED APPLICATION

This application claims priority to U.S. Provisional Application No. 61/249,415, which was filed on Oct. 7, 2009, the contents of which are incorporated herein by reference.

### FIELD OF THE INVENTION

The invention relates generally to prolonged-release drug formulations containing a blend of polymer and drug that are provided in the form of injectable microspheres. More specifically, the invention relates to microspheres that afford prolonged-release of anti-inflammatory glucocorticoids and other therapeutic agents.

### BACKGROUND OF THE INVENTION

Back pain is second only to cardiovascular disease as a cause of physical disability. Low back pain is also the most common reason for medical office visits and the most common reason for a worker's compensation claim. Upwards of 500,000 surgical spine procedures are performed each year in this country despite a complete lack of consensus of efficacy in improving long-term pain outcomes. The total annual cost to the U.S. economy from back pain has been estimated to be as high as $100 billion including more than $20 billion in direct medical expense.

The causes of chronic back pain are varied, complex, and often difficult to diagnose. In most cases, however, the patient is able to obtain some therapeutic relief without (or prior to) undergoing surgery. Providing the patient with pain relief without undergoing surgery is preferable in terms of reducing the escalation of costs that have placed our healthcare system in a state of crisis.

One cause of back pain that can be definitively diagnosed is osteoarthritis of facet joints, the pairs of tiny articulating surfaces between adjacent vertebrae. Rather than attempting to use imaging studies, which are often misleading, the physician injects a pain killer into the suspicious joint(s). If the pain subsides, then the offending joints will have been identified and the diagnosis confirmed. Once the patient obtains pain relief from injection, no matter how temporary, a great incentive exists to perform additional injections, both for the patient seeking repeat of a pain-free period and for the physician seeking to generate revenue.

Facet pain represents nearly 30% of all back pain conditions and yet lacks an effective, long term treatment. The short term relief obtained by injection of analgesic, mentioned above, can be extended in most cases by also injecting an anti-inflammatory steroid into the joint.

Although the cost of these anti-inflammatory steroids (referred to herein as “glucocorticoids”) is low because of their generic composition, the injection procedure itself is costly. This is because fluoroscopic guidance of hypodermic needle insertion is required prior to actual injection of the drug to ensure correct placement of the needle tip and (with the use of x-ray contrast fluid) that the drug will not inadvertently be injected into a blood vessel.

This procedure is typically performed on an out-patient basis at surgical centers. The high demand and high cost of this procedure, however, have created the need, satisfied by the present invention, for a longer-acting, prolonged time-release formulation of the injectable drug to reduce the number of procedures requiring medical expense reimbursement.

The most commonly prescribed glucocorticoids for use in facet joint injections are methylprednisolone (including methylprednisolone acetate, hereinafter “MPA”), betamethasone acetate and triamcinolone acetonide. Any co-injected analgesic would have a very short duration of action. Thus, the prior art teaches various chemical modifications of glucocorticoids to render them less water soluble as a means of slowing their bioavailability and prolonging the duration of residence in the desired location.

The injected dose is typically a suspension of comminuted crystalline particles suspended in an aqueous fluid. Currently available glucocorticoid products usually combine a water soluble form of the drug for immediate pain relief with suspended particles of the insoluble form for longer lasting effects, which typically amount to several weeks at best.

U.S. Pat. No. 7,157,102 entitled “Multi-layered Microcapsules and Method of Preparing Same,” by Nuwayser and assigned to Biotek, Inc. covers their anti-narcotic addiction product: Depotrex® Microcapsules (see: http://www.biotek-inc.com/depotrex.htm). This technology is related to the present invention in its objective of providing a diffusion rate-limiting encapsulant as a mechanism for controlled release. However, this prior art method of providing a prolonged-release formulation is restricted to the use of naltrexone as a drug and methylene chloride as a solvent for polymer processing.

Methylene chloride is a toxic and potentially carcinogenic substance that is difficult quantitatively to remove from polymers that are processed by being dissolved in this solvent. Thus, it is an objective of the present invention to provide a microsphere production process that does not require the use of toxic, chlorinated solvents.

U.S. Pat. No. 5,700,485 entitled “Prolonged Nerve Blockade by the Combination of Local Anesthetic and Glucocorticoid” by Berde and Langer represents a further attempt in the prior art to provide prolonged release of drug from polymer in the form of microspheres. However, it can be seen from the data presented that release rates of the active ingredients from the patented formulations are far too rapid to meet the requirements of the prolonged-release product of the present invention.

Moreover, the patented formulation of Berde & Langer exhibits a rapidly declining “first-order” release rate. This type of release rate is typically encountered by simply combining drug and polymer.

The first-order release rate is contrasted from a constant “zero-order” release rate that is generally viewed as desirable for long-term continuous release. Thus, it is a further objective of the present invention to provide microspheres of polymer and drug in which the drug is released at a relatively constant (i.e., “zero-order”) rate.

It is also well known in the art that a reasonably constant rate of drug release can be obtained from microspheres that contain only a very small amount of drug (i.e., low drug “loading”) and more difficult to achieve such rate with a high drug loading.

It is desirable to have as high a drug loading as possible so as not to require an excessive volume of injected microspheres into a site where physical space may be limited. Thus, another objective of the present invention is to provide a microsphere-drug system that not only provides a relatively constant rate of release, but also accommodates a high loading of drug within the microspheres.

The art of drug formulation technology is highly dependent on the availability of reliable in vitro test methods that simulate in vivo conditions and are predictive of in vivo performance. For example, a physiological buffer such as phosphate buffered saline solution can be used to extract the drug from the drug formulation at body temperature (37° C.), either under static (stirring) or dynamic (flow-through) conditions and analytically measuring the rate at which the drug partitions into the aqueous phase. In the case of MPA, however, such test methods of the prior art are impractical due to the extreme water insolubility of this compound.

Injectable MPA is sold as a suspension of particles in aqueous solution with no concern that the suspended particle will ever dissolve. Clearly the mechanism of MPA bioavailability in vivo involves more than simple dissolution. Most likely the interstitial tissue fluids surrounding injected MPA particles in vivo possess greater solvating power than standard laboratory inorganic aqueous buffers.

Thus, a further objective of the present invention is to provide an analytical test method that simulates MPA dissolution as it might occur in vivo and accelerates the time course of said dissolution such that comparisons of various potential prolonged release formulations can be made with convenience and reproducibility.

### SUMMARY OF THE INVENTION

An embodiment of the invention is based upon the discovery of a solvent-polymer pair that is non-toxic, pharmaceutically acceptable, and capable of facilitating the blending of MPA and related glucocorticoids with the polymer such that microspheres comprised of the drug-polymer blend are readily formed upon removal of the solvent. The resultant microspheres exhibit a surprisingly constant rate of drug release under the conditions of a novel in vitro release rate test designed specifically to accommodate the low water solubility of the preferred drugs.

The release rate is substantially independent of the quantity of drug blended in the polymer, within the composition limits required for microsphere formation. These drug-loaded microspheres provide the extended duration of constant in situ drug release required to address the clinical need for prolonged anti-inflammation therapy to alleviate chronic back pain via minimally invasive hypodermic injection.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other.

FIG. 1 is a light microscopy image of microspheres comprised of polycaprolactone (“PCL”) and methylprednisolone acetate (“MPA”).

FIG. 2 is a scanning electron microscopy image of microspheres comprised of PCL and MPA.

FIG. 3 is a particle size distribution plot of microspheres comprised of PCL and MPA.

FIG. 4 is a graph of the partition rate of N-methylpyrrolidone (“NMP”) from liquid droplets suspended in mineral oil at a constant stir speed into the oil phase upon fast addition (left line) versus slow addition (right line) of peanut oil to the mineral oil.

FIG. 5 is a schematic illustration of flow-through system for elution of MPA from PCL-MPA microspheres and from insoluble particles of MPA.

FIG. 6 are two photographs of glass frit filters packed with MPA and Celite (left) for 37° C. bath flow-through MPA elution and packed with 3 mm diameter glass beads (right) for 4° C. bath flow-through MPA deposition. Arrows show direction of eluant flow in the system.

FIG. 7 is a chart of a comparison of elution rates of MPA micronized powder and PCL microspheres containing about 10% and 20% MPA.

FIG. 8 is a mixing apparatus used for making microspheres.

FIG. 9 is a plot of MPA concentration in pure 1,4-dioxane versus absorbance at 295 nm.

### DETAILED DESCRIPTION OF THE PREFERRED EMBODIMENTS

An embodiment of the invention is directed to prolonged-release injectable steroids. The prolonged-release injectable steroids may be provided in the form of microspheres. The prolonged-release injectable steroid microspheres may include a polymer, a drug and a solvent.

The microspheres are injected into the patient for the treatment of pain caused by osteoarthritis in at least one of the patient's facet joint. A person of skill in the art will appreciate that it is possible to use the microspheres in other applications.

One suitable polymer for use in conjunction with the invention is polycaprolactone (“PCL”) because this material has a history of safety as a bioabsorbable implant material and commercial availability. In addition, PCL was selected based upon its known slow rate of in vivo degradation and bioabsorption. Thus a desirable feature of the present invention is that the time course of drug release is not required to coincide with the time course of polymer disintegration.

Although examples of polymer-drug compositions have been described in which the mechanism of drug release is tied to the rate of polymer degradation or surface erosion, the invention benefits from the discovery that the drug is capable of molecular migration through the polymer. Without being bound by any theory, it appears that the drug diffuses through the bulk of the microsphere and thereafter is eluted from its surface as a final rate-limiting mechanism of release.

The choice of synthetic bioabsorbable polymers that are useful in the invention is limited by the relatively small number of such polymers commercially available and clinically acceptable for pharmaceutical applications. These polymers were originally utilized in the development of absorbable surgical suture. This list includes polymers made from one or more of the following monomers: lactide, glycolide, caprolactone, dioxanone, and trimethylene carbonate.

However, the number of possible combinations and permutations of the preceding monomers is quite larger. In general, polymers that are useful in the present invention have low crystallinity, low glass transition temperature, and a slow rate of bioabsorption. Thus, copolymers (including block copolymers, random copolymers, and polymer blends) containing a high content of polycaprolactone and/or poly(trimethylene carbonate) are preferred. Based upon the prior work that has been done and the associated literature with the polymers set forth above, it is envisioned that the preceding polymers would have similar performance to PCL.

One suitable classification of drugs for use in conjunction with the invention are steroids, which are intended for injection to treat pain that is being experienced in portions of a human body. One such suitable steroid is methylprednisolone, which is the generic name for 11β,17α,21-trihydroxy-6α-methylpregna-1,4-diene-3,20-dione and has the same actions and uses as 6-methylprednisolone 21-acetate (MPA). This steroid is particularly suitable for intrasynovial and soft-tissue injection.

In addition to MPA (Depo-Medol; Pharmacia-Upjohn, Kalamazoo, Mich.), other steroids may be incorporated into the microspheres. Some examples of other suitable steroids are triamcinolone acetonide (Kenalog; Bristol-Meyers Squibb, Princeton, N.J.), dexamethasone sodium phosphate (Decadron; American Regent Laboratories, Shirley, N.J.), betamethasone sodium phosphate/betamethasone acetate (Celestone Soluspan; Schering-Plough, Kenilworth, N.J. or betamethasone repository; New England Compounding Center, Framingham, Mass.). Based upon the prior work that has been done and the associated literature with the steroids set forth above, it is envisioned that the preceding steroids would have similar performance to MPA.

In certain embodiments, the solvent is 1-methyl-2-pyrrolidinone (herein referred to as “NMP” for its more common name of N-methylpyrrolidone). Related solvents in terms of their solvent power could also be used, for example dimethylsulfoxide, formamide, and dimethyl formamide, especially if provided in anhydrous grade.

NMP is believed to be an advantageous choice based upon its combination of solvent properties and toxicological safety. The latter feature is important because it is anticipated that any such processing solvent will be analytically detectable as a trace residue in the finished microspheres. Based upon the prior work that has been done and the associated literature with the solvents set forth above, it is envisioned that the preceding solvents would have similar performance to NMP.

The polymer may be provided as pellets that are solid at ambient temperature. Because the polymer may have only limited solubility in the solvent at ambient temperature, it may be desirable to heat the polymer to cause the polymer to melt and thereby increase the solubility of the polymer in the solvent. In certain embodiments, the polymer is heated to a temperature of at least about 60° C. and then mixed with the solvent. A person of skill in the art will appreciate that different temperatures may be used to provide good solubility of the polymer in the solvent.

After heating, the polymer pellets may change from white to clear, presumable due to loss of crystallinity. If allowed to cool without mixing, the pellets may regain their white appearance. Upon stirring, the clear pellets dissolve to form a viscous solution with the solvent that remains clear upon cooling to room temperature.

Next, the drug is mixed with the polymer-solvent solution. It was found that the drug, which was provided as a solid, was soluble when mixed into the polymer-solvent solution. The mixing was continued until the solution was substantially homogeneous.

In certain embodiments, the microspheres may be fabricated from a solution of solvent (NMP) containing dissolved drug and polymer by a process that is surprisingly simple. This process is based upon the discovery that solvent (NMP) is immiscible in a first oil and miscible in a second oil. In certain embodiments, the first oil is mineral oil and the second oil is vegetable oil.

Using this procedure, the drug-polymer-solvent solution is suspended in mineral oil with mixing to produce microdroplets of the former in the later. Vegetable oil, for example peanut oil, is then added to the mix with continued stirring.

Since mineral oil and peanut oil are miscible with each other, this resultant change in the composition of the oil phase causes the NMP to partition out of the suspended droplets and into the oil phase. This process thereby leaves the remaining polymer-drug in the form of solid microspheres.

The microspheres may then be collected on a filter. After collection on a filter, the microspheres may be rinsed to remove any remaining oil. An example of a suitable liquid that may be used in conjunction with the rinse is a hydrocarbon solvent such as hexane.

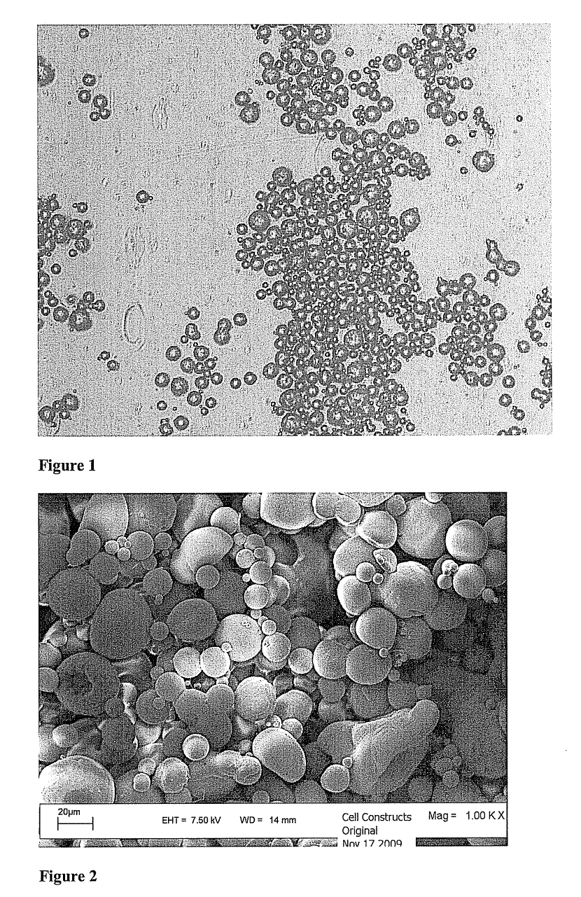

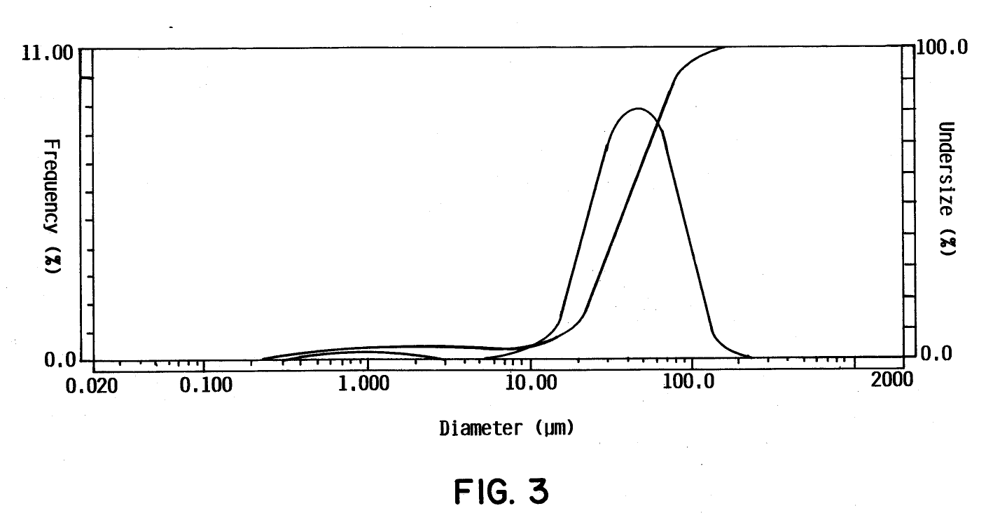

Light and scanning electron photomicrographs of typical microspheres of the present invention are illustrated in FIGS. 1 and 2, respectively. A typical result for particle size distribution of the microspheres is illustrated in FIG. 3. A high percentage of the microspheres have a diameter of between about 10 micrometers and about 100 micrometers. The median size of the microspheres is about 40.8 micrometers with a standard deviation of about 26.9 micrometers.

In certain embodiments, the volume ratio of oils in the final mix after addition of peanut oil is between about 1:3 and 1:7 mineral oil to peanut oil. In other embodiments, the ratio of mineral oil to peanut oil may be about 1:5. The preceding ratio ensures substantially complete abstraction of the solvent from the droplets and into the oil phase.

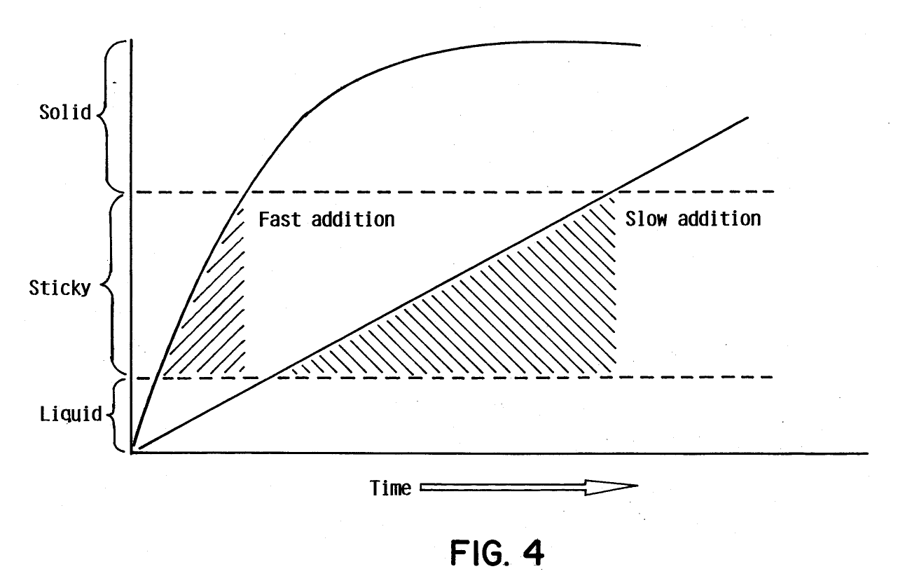

A further observation was that rapid addition of peanut oil gives a higher yield of microspheres. It is believed that the rapid addition of peanut oil limits the time that the microsphere composition is in transition from liquid to solid and thereby reduces the number of microspheres that agglomerate due to stickiness, as schematically illustrated in FIG. 4.

Other methods of making the microspheres of the present invention are anticipated. One such suitable process involves spraying droplets of the polymer-drug-solvent solution directly into an oil in which the solvent is miscible such as peanut oil. This process obviates the need for mineral oil completely.

This spraying process may be accomplished with the use of equipment that has been specifically developed for coagulation of liquids into beads. An example of one such suitable system that may be used for formation of the microspheres is an aerodynamically assisted jetting device. An advantage of this type of system is that it enables microspheres to be produced with precisely controlled size distribution. An example of such equipment is the Nisco Encapsulation Unit (Var-J30), made by Nisco Engineering AG, Switzerland.

In other embodiments, the invention relates to a test method that is used to characterize the microspheres with respect to potential in vivo rate of drug release by comparing their in vitro drug release rate to control drug particles. Typically, the most convenient method of quantitative analysis of MPA is by its ultraviolet absorbance, which requires the use of a standard laboratory spectrophotometer.

This analytical method has several drawbacks that preclude its use in conjunction with evaluating the concentration of eluted MPA directly in the eluant from microspheres produced according to this invention. For example, this prior method of direct detection requires that the eluant in which the MPA is dissolved to be UV transparent in the spectral region of interest. A further requirement is that the concentration of MPA is not too low for detection.

Since the goal is to have a test with some measure of approximation to the in vivo environment, it is desirable to conduct the elution in an aqueous system. Because of the extremely low water solubility of MPA, it is not possible to use pure water as a medium for in vitro evaluation of drug elution.

Although many possibilities exist for simulation of in vivo elution, such as use of human blood serum, it is undesirable to use materials that contain protein as such protein will interfere with the UV detection of MPA. The use of water miscible organic solvents to increase the solubility of MPA in water is a practical solution to this problem. However, the solvent not only must be UV transparent but also must not cause swelling or dissolution of the PCL polymer.

It was discovered that a solution of 1,4-dioxane and water meets the preceding requirements. In certain embodiments, the 1,4-dioxane is mixed with water at a ratio of between about 1:3 and 1:5. In other embodiments the ratio of 1,4-dioxane to water is about 1:4.

In an effort to replicate the conditions under which the microspheres will be subjected to when used in a patient, this solution was maintained at a temperature of between about 30° C. and about 45° C. In certain embodiments, the solution was maintained at a temperature of about 37° C. to replicate the temperature of a typical human body.

The test method of the invention may include a flow through system in which the microspheres reside on a glass frit filter that is maintained at a temperature of about 37° C. using a heated water bath. The eluant comprised of a mixture of water and 1,4-dioxane is recirculated through the microspheres using a pump such as a peristaltic pump.

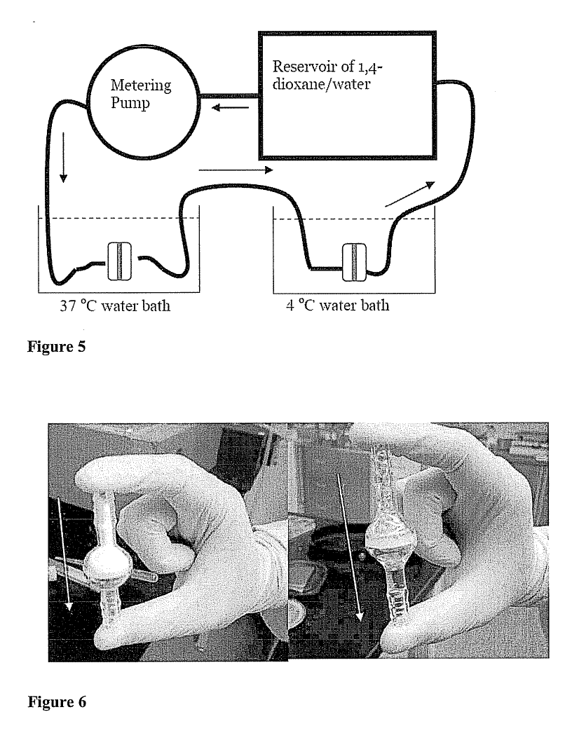

Prior to recirculation through the temperature controlled bath, the eluant may be passed through a column of glass beads on another glass frit filter in a low temperature controlled bath, as schematically illustrated in FIG. 5.

In certain embodiments, the low temperature controlled bath was maintained at a temperature of less than about 10° C. In other embodiments, the low temperature controlled bath was maintained at a temperature of about 4° C. This process causes any MPA in the eluant to collect on the low temperature beads such that the eluant that is recirculated to the microspheres is substantially free of MPA.

With this system running, the filter containing the glass beads can be replaced periodically with a filter containing fresh glass beads to measure the amount of MPA that is released from the microspheres. Over time an imperceptive thin film of MPA becomes deposited on the glass beads. After a selected period of time, the MPA film may be rinsed off with pure dioxane and assayed for MPA content with the use of ultraviolet spectrophotometry via absorbance at 295 nm.

In this test method, the use of pure MPA as a reference material was complicated by its tendency to pack into hard lumps under conditions of the test. To prevent packing of the MPA, diatomaceous earth was mixed with the MPA. A similar volume of diatomaceous earth was also added to test samples of the microspheres. An example of the elution and collection filters containing test material and glass beads, respectively, is illustrated in FIG. 6.

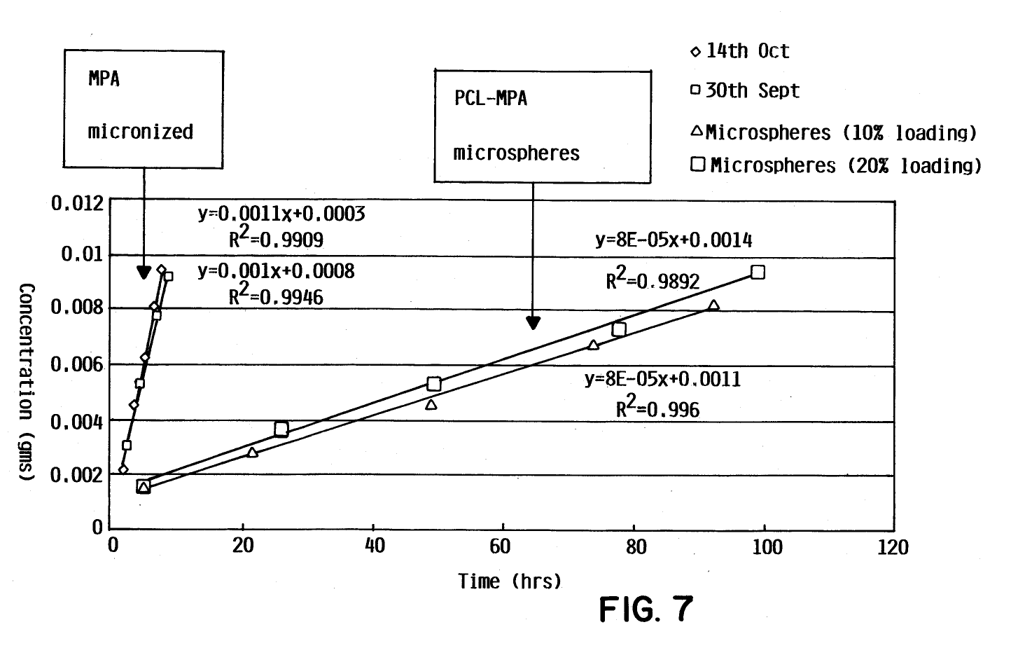

As illustrated in FIG. 7, the in vitro elution test results demonstrated an unexpectedly desirable and useful prolonged release rate of MPA from microspheres in comparison to control MPA particles. Surprisingly, a doubling of the drug content within the microspheres had virtually no effect on the rate of drug release.

The product and method of the present invention are described in the following examples. These examples are provided as an illustration of the invention and are not intended to limit the invention.

### Example 1

### Microsphere Manufacturing Process

The materials used in these manufacturing process experiments are listed in Table 1.

TABLE 1

| Material | Name | Vendor | Catalog No. | Lot No. |
|---|---|---|---|---|
| Polymer | Polycaprolactone | Sigma- | 440744 | 00807DJ |
| | (PCL) | Aldrich | | |
| Solvent | 1-Methyl-2- | Sigma- | 328634 | 73196EJ |
| | pyrollidinone | Aldrich | | |
| | (NMP), anhydrous | | | |
| Drug | Methylprednisolone | Spectrum | ME171 | WH1073 |
| | acetate (MPA) | | | |
| Oil | Peanut oil | Kroger | (LouAnn/Ventura | Z0348 |
| | | | Foods, LLC) | |
| Oil | Mineral oil, USP | Wal-Mart | (Cumberland | SHF0692 |
| | | | Swan) | |
| Rinse | Hexane | Sigma- | 208752 | 62896HJ |
| solvent | | Aldrich | | |

The polymer (PCL) was obtained in the form of white pellets that were heated to a temperature of about 60° C. and then mixed with the solvent (NMP) to form a polymer-solvent solution.

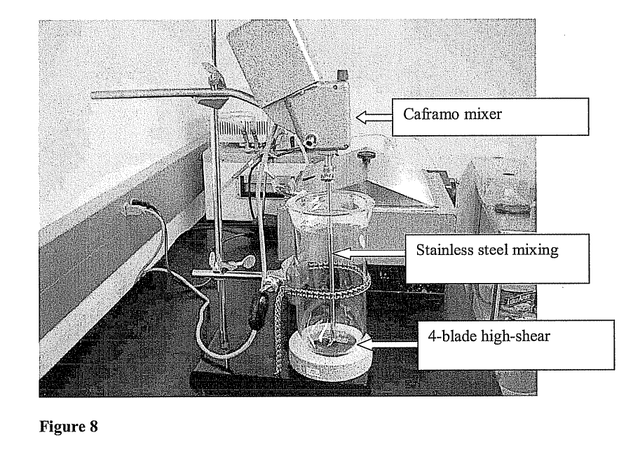

The drug (MPA), which is soluble in NMP, was mixed as a solid into the PCL/NMP solution. The resultant drug-polymer-NMP solution was poured into stirring mineral oil. The equipment used for mixing consisted of a 2-liter resin flask and a motor-driven stainless steel impeller, as illustrated in FIG. 8.

Peanut oil was then added to the stirring mixture and stirring allowed to continue for about one hour. The stirring speed utilized was about 90 rpm. The product was collected by filtration, rinsed free of oil with hexane and stored in tightly sealed glass vials prior to evaluation. The quantities of materials used in two typical batches are shown in Table 2.

TABLE 2

| | Quantities Used | | | | | | |
|---|---|---|---|---|---|---|---|
| | | | | | Peanut | | |
| | PCL | NMP | MPA | Mineral | Oil | Percent | Percent |
| Batch | (gms) | (mL) | (gms) | Oil (mL) | (mL) | Drug | Yield |
| 1 | 0.8001 | 20 | 0.0890 | 100 | 500 | 11.1% | 52% |
| 2 | 1.636 | 40 | 0.1663 | 200 | 1000 | 10.2% | 60% |

### Example 2

### In Vitro MPA Elution from MPA Particles and MPA-PCL-Microspheres

To run the control elution test, about 50 mg of MPA were mixed with about 200 mg of diatomaceous earth (Celite® from Sigma-Aldrich) and placed in a fritted glass disc filter, which was maintained at a temperature of about 37° C. A similar filter was packed with 3 mm diameter glass beads to serve as the collection filter, which was maintained at a temperature of about 4° C.

As illustrated in FIG. 5, silicone rubber tubing was connected from a bottle containing approximately 300 ml of a mixture of water and 1,4-dioxane (1:4 by volume) to a peristaltic pump (Manostat® from Fisher Scientific). The tubing from the pump was then connected to the sample filter, which contained the test material and diatomaceous earth. Next, from the exit of the sample filter the tubing was connected to the entrance of the collection filter containing the glass beads. Finally, the tubing was connected from the exit of the collection filter back to the reservoir bottle of water and 1,4-dioxane to complete the recirculation loop.

With the water bath heaters and refrigerators set and the water pre-equilibrated to the proper temperatures, the filters were immersed in the water and the pump was started. The pump was operated at a flow rate of 200 ml per hour.

To assay accumulated MPA, the pump was stopped and the collection filter was removed from the system. The glass beads were removed from the collection filter for analysis. Fresh glass beads were placed in the collection filter and the collection filter was reconnected and the pump re-started. This process was done in a relatively short period of time.

The various batches of recovered beads were allowed to dry and placed in a vial containing a precisely measured volume of pure 1,4-dioxane. The 1,4-dioxane dissolved the deposits of MPA collected the surfaces of the glass beads.

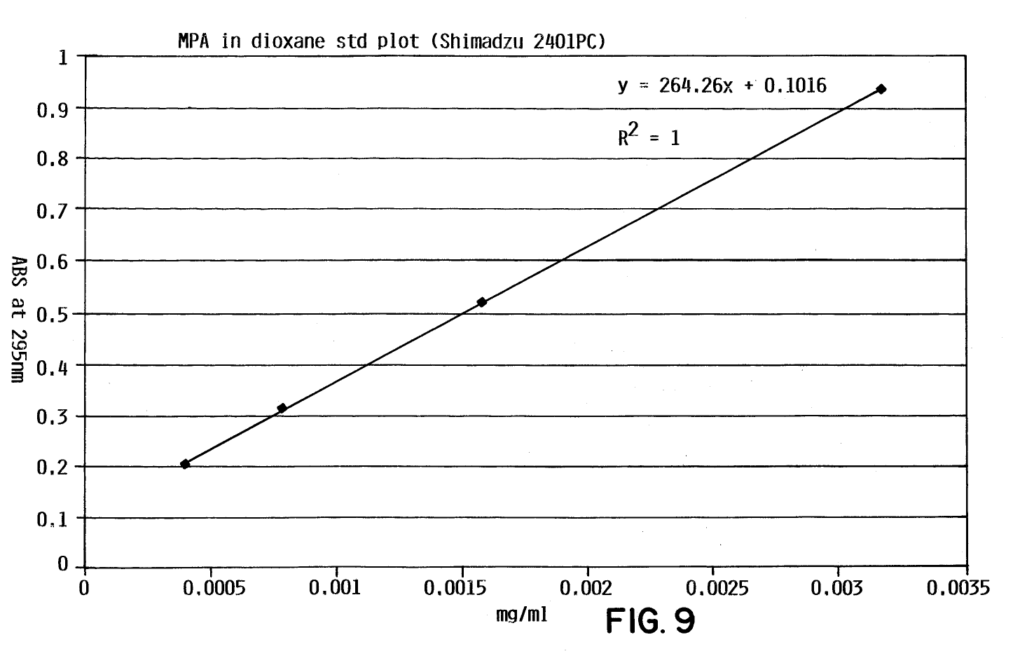

An aliquot of this solution of MPA in 1,4-dioxane was placed in a quartz cuvette and the absorbance at 295 nm measured in a spectrophotometer. The concentration of MPA was determined from a standard plot as illustrated in FIG. 9.

The amount of MPA collected on the beads was calculated by multiplying the concentration of MPA in the 1,4-dioxane times the volume of 1,4-dioxane in which the glass beads were immersed. This procedure was repeated for each time point corresponding to a change of glass bead-packed filters.

The above experiment was performed twice on separate dates using the microspheres of Example 1. Another batch of microspheres prepared by the same process but with 20% drug loading also was evaluated twice. The results of all these tests are shown in FIG. 6, which compares the MPA release rate from pure MPA particles with the release rate of MPA from the microspheres of the present invention. As can be readily seen, the release of MPA from microspheres occurs over a period of time that is approximately 10 times longer than with MPA particles alone.

While preferred illustrative embodiments of the invention are described above, it will be apparent to one skilled in the art that various changes and modifications may be made therein without departing from the invention and it is intended in the appended claims to cover all such changes and modification which fall within the true spirit and scope of the invention.

1. A method of forming prolonged-release injectable steroids comprising:
providing a steroid composition;
providing a bioabsorbable polymer;
providing a solvent;
forming a solution from the steroid composition, the bioabsorbable polymer and the solvent;
forming droplets from the solution; and
removing the solvent from the droplets to cause the droplets to form microspheres.

2. The method of claim 1, wherein the steroid composition comprises methylprednisolone acetate, triamcinolone acetonide, dexamethasone sodium phosphate, betamethasone sodium phosphate, betamethasone acetate and combinations thereof.

3. The method of claim 1, wherein the bioabsorbable polymer is fabricated from one or more of the following monomers: lactide, glycolide, caprolactone, dioxanone, and trimethylene carbonate.

4. The method of claim 1, wherein the solvent comprises N-methylpyrrolidone, dimethylsulfoxide, formamide, dimethyl formamide or combinations thereof.

5. The method of claim 1, wherein the solvent has a high ability to dissolve the bioabsorbable polymer and the steroid composition and wherein the solvent has a low human toxicology.

6. The method of claim 1, wherein the solvent is immiscible in a first oil and miscible in a second oil and wherein forming the droplets from the solution comprises suspending the solution in the first oil to produce droplets.

7. The method of claim 6, wherein removing the solvent from the droplets to form the microspheres comprises:
adding the second oil to the first oil and the solution to form a mixture;
stirring the mixture;
removing the solvent from the droplets to form microspheres; and
collecting the microspheres on a filter.

8. The method of claim 7, and further comprising rinsing the microspheres with a second solvent.

9. The method of claim 8, wherein the second solvent is hexane.

10. The method of claim 7, wherein a ratio of the first oil to the second oil is between about 1:3 and 1:7.

11. The method of claim 7, wherein the second oil is rapidly added to the first oil.

12. The method of claim 7, wherein the first oil is mineral oil.

13. The method of claim 7, wherein the second oil is vegetable oil.

14. The method of claim 1, wherein the microspheres have a diameter of between about 10 micrometers and about 100 micrometers.

15. The method of claim 1, wherein the forming droplets from the solution and the removing the solvent from the droplets to cause the droplets to form microspheres spraying droplets of the solution into a first oil to produce microspheres.

16. The method of claim 15, wherein spraying the droplets is done using an aerodynamically assisted jetting device.

17. Prolonged-release injectable steroids comprising a microsphere comprising a steroid composition and a bioabsorbable polymer, wherein the steroid composition is released from the bioabsorbable polymer at a relatively constant rate that is substantially independent of a concentration of the steroid composition in the bioabsorbable polymer.

18. The prolonged-release injectable steroids of claim 17, wherein the steroid composition comprises methylprednisolone acetate, triamcinolone acetonide, dexamethasone sodium phosphate, betamethasone sodium phosphate, betamethasone acetate and combinations thereof.

19. The prolonged-release injectable steroids of claim 17, wherein the bioabsorbable polymer has a low crystallinity, low glass transition temperature, and a slow rate of bioabsorption.

20. The prolonged-release injectable steroids of claim 17, wherein the bioabsorbable polymer is fabricated from one or more of the following monomers: lactide, glycolide, caprolactone, dioxanone, and trimethylene carbonate.

21. The prolonged-release injectable steroids of claim 17, wherein the solvent comprises N-methylpyrrolidone, dimethylsulfoxide, formamide, dimethyl formamide or combinations thereof.

22. The prolonged-release injectable steroids of claim 17, wherein the solvent has a high ability to dissolve the bioabsorbable polymer and the steroid composition and wherein the solvent has a low human toxicology.

23. The prolonged-release injectable steroids of claim 17, wherein the solvent is immiscible in a first oil and miscible in a second oil and wherein forming the droplets from the solution comprises suspending the solution in the first oil to produce droplets.

24. The prolonged-release injectable steroids of claim 17, wherein the microspheres have a diameter of between about 10 micrometers and about 100 micrometers.

25. A method of simulating drug release from a microsphere comprising:
preparing an eluant solution from 1,4-dioxane;
flowing the eluant solution past a plurality of microspheres containing a steroid composition and a bioabsorbable polymer;
eluting the drug composition from the microspheres into the eluant solution; and
collecting the eluted steroid composition.

26. The method of claim 26, wherein the steroid composition comprises methylprednisolone acetate, triamcinolone acetonide, dexamethasone sodium phosphate, betamethasone sodium phosphate, betamethasone acetate and combinations thereof.

27. The method of claim 26, wherein the bioabsorbable polymer is fabricated from one or more of the following monomers: lactide, glycolide, caprolactone, dioxanone, and trimethylene carbonate.

28. The method of claim 26, wherein the eluant solution further comprises water and the volume ratio of 1,4-dioxane to water is between about 1:3 to about 1:5.

29. The method of claim 26, and further comprising maintaining the microspheres at a temperature of between about 30° C. and about 45° C.

30. The method of claim 26, wherein collecting the steroid composition is by flowing the eluant solution with the eluted steroid composition past a plurality of glass beads.

31. The method of claim 31, wherein the glass beads are maintained at a temperature of less than about 10° C.
